(19) United States
(12) Patent Application Publication
(10) Pub. No.: US 2019/0254947 A1
(43) Pub. Date: Aug. 22, 2019

(54) FILM-FORMING COMPOSITION FOR SKIN OR HAIR COATING

(71) Applicant: AMOREPACIFIC CORPORATION, Seoul (KR)

(72) Inventors: Yoon Kyun HWANG, Yongin-si, Gyeonggi-do (KR); Sung Ah BIN, Yongin-si, Gyeonggi-do (KR); Da Woon AHN, Yongin-si, Gyeonggi-do (KR); Jang Won CHOI, Yongin-si, Gyeonggi-do (KR); Jong Hyub KIM, Yongin-si, Gyeonggi-do (KR); Sung II PARK, Yongin-si, Gyeonggi-do (KR)

(73) Assignee: AMOREPACIFIC CORPORATION, Seoul (KR)

(21) Appl. No.: 16/337,248

(22) PCT Filed: Sep. 26, 2017
(86) PCT No.: PCT/KR2017/010577

§ 371 Date: Mar. 27, 2019

(30) Foreign Application Priority Data
Sep. 27, 2016 (KR) 10-2016-0123968
Sep. 25, 2017 (KR) 10-2017-0123468

(51) Int. Cl. A61K 8/58; A61K 8/892; A61K 8/06; A61K 8/86; A61Q 19/00; A61Q 5/00

(52) U.S. Cl. CPC A61K 8/585; A61K 8/892; A61Q 5/00; A61K 8/86; A61Q 19/00; A61K 8/062

(57) ABSTRACT

The present invention relates to a film-forming composition for skin or hair coating. The film-forming composition for skin or hair coating according to the present invention exhibits a feeling of lightness when applied to skin or hair and has excellent durability to protect skin or hair itself, and also, can protect cosmetic applications to the skin, etc. Also, the present invention separates and prepares silane and water so that a film is formed at the time of application, thereby being convenient in use.

### TECHNICAL FIELD

This application claims the benefits of priorities based on Korean Patent Application No. 10-2016-0123968, filed on Sep. 27, 2016 and Korean Patent Application No. 10-2017-0123468, filed on Sep. 25, 2017, the entire contents of which are incorporated herein by reference.

The present invention relates to a film-forming composition for skin or hair coating.

### BACKGROUND ART

There is a phenomenon that cosmetics and the like applied to the skin of a person are gradually detached and disappeared from the skin as time passes after application and the makeup becomes thin. Also, the skin itself, made of proteins, can be damaged by external factors.

In addition, human hair includes spiral proteins known as keratin, like animal hair, horns, claws, skin and feathers. The proteins of this structure are degraded as they continue to be exposed to external forces, sunlight, harsh chemicals, and contaminants in air, which can lead to hair damage.

The physical properties of damaged skin or hair, etc. are different from those of healthy skin or hair. In order to protect them, a cosmetic product may be used to form a protective film on skin or hair. However, the retentiveness and retention time of the film can be achieved only when the molecular weight of the material forming the film is higher than a certain level, and the larger the molecular weight, the thicker the film becomes, thereby resulting in stuffiness and heavy feeling of use. However, when the film is formed using a low-molecular material, it may exhibit strong volatility, or the film-forming itself may be difficult.

Therefore, there is an urgent need to study a technique for forming a film using a low-molecular material.

### PRIOR ART DOCUMENT

### Patent Document

(Patent Document 1) Korean Laid-open Patent Publication No. 2006-0035343, “Hair coating composition comprising polyethyleneglycol derivatives as an effective ingredient” and

(Patent Document 2) Korean Laid-open Patent Publication No. 2016-0108126, “Vegetable hair protective agent, its preparation method and coating method.”

### DISCLOSURE

### Technical Problem

The retentiveness and retention time of the existing film for protecting skin or hair can be achieved only when the molecular weight of the film forming material is higher than a certain level, but the larger the molecular weight, the thicker the film becomes, thereby resulting in a stuffiness and heavy feeling of use. However, the low molecular weight materials have the disadvantage that they are difficult to form a film and have low durability.

Accordingly, it is an object of the present invention to provide a film-forming composition using a low-molecular material having a light feeling of use and an excellent durability.

### Technical Solution

In order to achieve the above object, the present invention provides a film-forming composition for skin or hair coating comprising silane and water, wherein the silane is water-dispersed.

At this time, the film-forming composition has a form in which a silane carrying micelle is water-dispersed, which includes the silane and water separately, and may form a film by reacting the silane with the water when applied.

At this time, the silane may be at least one silane compound selected from the group consisting of aminopropyltriethoxysilane, hexyltriethoxysilane, perfluorooctyltriethoxysilane, triethoxycaprylylsilane, retinoxytrimethylsilane, stearyltriethoxysilane and stearoxytrimethylsilane.

At this time, the silane may be included in an amount of 0.1 to 5 wt. % based on the total weight of the film-forming composition.

At this time, the film-forming composition may further comprise a silicone-based oil.

At this time, the silicone-based oil may be included in an amount of 1 to 40 wt. % based on the total weight of the film-forming composition

At this time, the weight-average molecular weight of the silicone-based oil may range from 3,000 to 50,000.

At this time, the silicone-based oil may be at least one selected from the group consisting of dimethiconol, dimethicone, cyclomethicone, cyclophenylmethicone, capryldimethicone, caprylyltrimethicone, caprylylmethicone, cetearylmethicone, hexadecylmethicone, hexylmethicone, laurylmethicone, myristylmethicone, phenylmethicone, stearylmethicone, stearyldimethicone, trifluoropropylmethicone, cetyldimethicone, polyphenylmethylsiloxane, dimethylpolysiloxane, methylphenylpolysiloxane, cyclopentasiloxane, decamethylcyclopentasiloxane, cyclohexasiloxane, cyclohepatsiloxane, cyclotetrasiloxane, cyclotrisiloxane, methyltrimethicone, diphenylsiloxyphenyltrimethicone, caprylylmethicone and phenyltrimethicone.

At this time, the film-forming composition may further comprise a surfactant and may be an emulsion in which micelles carrying the silane compound in the core are water-dispersed.

At this time, the surfactant may be at least one cationic surfactant selected from the group consisting of steartrimoniumchloride, cetrimoniumchloride, behenyltrimethylammonium chloride, cetyltrimethylammonium chloride, stearyltrimethylammonium chloride, distearyldimethylammonium chloride, dicetyldimethylammonium chloride, cocamidopropyldimethylamine, stearamidopropyldimethylamine, behenylamidopropyldimethylamine, oleamidopropyldimethylamine and isostearamidopropyldimethylamine; or may be at least one non-ionic surfactant selected from the group consisting of Trideceth-10, Trideceth-12, lauryl glucoside, decyl glucoside, PEG-20 glyceryl triisostearate, PEG-15 glyceryl isostearate, polyglyceryl-3 diisostearate and polyglyceryl-10 dioleate.

At this time, when the film-forming composition is applied to skin or hair, the micelle is broken and the silane and the water react with each other to form a film.

### Advantageous Effects

The film-forming composition for skin or hair coating according to the present invention exhibits a light feeling of use when applied to skin or hair and has excellent durability to protect skin or hair itself, and also, can protect cosmetic applied to the skin, etc.

Also, since the film-forming composition has a form in which micelles containing silane are water-dispersed, before application to skin or hair, silane and water exist separately, and then when applied to skin or hair, the micelle is broken and at the same time the silane and water reacts with each other to form a film, thereby being convenient in use and exhibiting excellent coating effect.

### DESCRIPTION OF DRAWINGS

FIG. 1 shows a process of measuring the contact angle of Experimental Example 1.

FIG. 2 shows the measurement results of the water repellency of the hair surface through the measurement of contact angle of Experimental Example 1

FIG. 3 shows the results of the evaluation of the durability of the film-forming composition of Experimental Example 2.

### BEST MODE

Hereinafter, in order to describe the present invention more specifically, preferred embodiments according to the present invention will be described. However, the present invention is not limited to the embodiments described herein but may be embodied in other forms.

The present invention relates to a film-forming composition for skin or hair coating, characterized in that it comprises silane and water, which has excellent coating effect on skin or hair even though a raw material having low molecular weight is used in order to form a thin film to prevent heavy feeling of use when applied to skin or hair.

In addition, the film-forming composition includes the silane and the water separately and thus when applied to skin or hair, the silane and the water can react with each other to form a film.

The term “film-forming composition for skin or hair coating” as used herein may refer to a film-forming composition for coating either skin or hair, or may refer to a film-forming composition for coating both skin and hair.

Hereinafter, each of the main components used in the preparation in the present invention will be described in more detail.

Silane

If silane reacts with water, alkoxy groups in silane are hydrolyzed to form silanol groups. The silanol group can condense with other silanol group to form a siloxane polymer chain and thus form a film. If the film formed by the silane and water is formed on skin or hair, it acts as a low molecular weight protective film to protect the skin and hair. Also, when a cosmetic product is applied to skin or a dye or the like is applied to hair and then the film is formed thereon, makeup or dyeing durability and the like can be improved. In addition, if silane is used to form a film, a low molecular film can be formed, thereby providing a light feeling of use.

The silane is a mono-molecular material, and may be at least one selected from the group consisting of aminopropyltriethoxysilane, hexyltriethoxysilane, perfluorooctyltriethoxysilane, triethoxycaprylylsilane, retinoxytrimethylsilane, stearyltriethoxysilane and stearoxytrimethylsilane.

In the present invention, the silane may be contained in an amount of 0.1 to 5 wt. % based on the total weight of the film-forming composition. If the content of the silane is less than the above range, it is difficult to form a film, so that the coating effect on the skin or hair is deteriorated. If the content of the silane exceeds the above range, the feeling of use is heavy and thus the preference level may be lowered.

Silicone-Based Oil

In the present invention, the film-forming composition may further comprise a silicone-based oil. The silicone-based oil is added to the film-forming composition to impart softness and play a role in protecting skin or hair.

The weight-average molecular weight of the silicone-based oil may range from 3,000 to 50,000. If the weight-average molecular weight of the silicone-based oil is less than 3,000, the strength of the film during film-forming on the skin or hair is lowered and the effect of improving the softness of the composition is insignificant. If the weight-average molecular weight of the silicone-based oil exceeds 50,000, the effect of improving the softness of the composition may be insignificant and the skin and hair protecting function may be deteriorated.

At this time, the silicone-based oil may be at least one selected from the group consisting of dimethiconol, dimethicone, cyclomethicone, cyclophenylmethicone, capryldimethicone, caprylyltrimethicone, caprylylmethicone, cetearylmethicone, hexadecylmethicone, hexylmethicone, laurylmethicone, myristylmethicone, phenylmethicone, stearylmethicone, stearyldimethicone, trifluoropropylmethicone, cetyldimethicone, polyphenylmethylsiloxane, dimethylpolysiloxane, methylphenylpolysiloxane, cyclopentasiloxane, decamethylcyclopentasiloxane, cyclohexasiloxane, cyclohepatsiloxane, cyclotetrasiloxane, cyclotrisiloxane, methyltrimethicone, diphenylsiloxyphenyltrimethicone, caprylylmethicone and phenyltrimethicone, and preferably may be dimethiconol.

The silicone-based oil may be contained in an amount of 1 to 40 wt. % based on the total weight of the film-forming composition. If the content of the silicone-based oil is less than the above range, the feeling of application on the skin is lowered and the effect of improving the softness of composition is insignificant. If the content exceeds the above range, the skin and hair protecting function may be deteriorated.

Surfactant

The film-forming composition may further comprise a surfactant and may be an emulsion in which micelles carrying the silane compound in the core are water-dispersed.

Surfactant is usually present in the form of a molecule in aqueous solution, but as the concentration of the surfactant in the solution increases, the surface tension decreases. The film-forming composition may further comprise a surfactant and may be an emulsion in which micelles carrying the silane compound in the core are water-dispersed.

Surfactant is usually present in the form of a molecule in aqueous solution, but as the concentration of the surfactant in the solution increases, the surface tension is deteriorated, and then if the deterioration of the surface tension becomes more difficult to occur, the hydrophobic groups of the surfactant are brought into association with each other to form a micelle, which is an agglomerate having a structure in which the hydrophobic group is directed toward the inside and the hydrophilic group is directed toward the outside so as to be in contact with water. The concentration of the surfactant solution that forms the micelle on this wise is called critical micelle concentration (CMC). Before and after the critical micelle concentration, physical properties such as interfacial tension, surface tension, electrical conductivity, cleaning ability, and viscosity, etc. of the surfactant solution are drastically changed. For example, above the critical micelle concentration, the surfactant solution has the property of dissolving a third substance, which does not dissolve in the surfactant solution, into the micelle, and this property is called solubilization.

When a film-forming composition in an emulsion state in which a mixture of silane or silane and silicone oil is carried in the core of the micelle and the micelle is dispersed in water is prepared, the silane and water can be present in an unreacted state.

In addition, the surfactant may be at least one cationic surfactant selected from the group consisting of steartrimoniumchloride, cetrimoniumchloride, behenyltrimethylammonium chloride, cetyltrimethylammonium chloride, stearyltrimethylammonium chloride, distearyldimethylammonium chloride, dicetyldimethylammonium chloride, cocamidopropyldimethylamine, stearamidopropyldimethylamine, behenylamidopropyldimethylamine, oleamidopropyldimethylamine and isostearamidopropyldimethylamine; or at least one non-ionic surfactant selected from the group consisting of Trideceth-10, Trideceth-12, lauryl glucoside, decyl glucoside, PEG(Poly Ethylene Glycol)-20 glyceryl triisostearate, PEG-15 glyceryl isostearate, polyglyceryl-3 diisostearate and polyglyceryl-10 dioleate.

The concentration of the surfactant in the film-forming composition of the present invention may be higher than the critical micelle concentration, and the diameter of the micelle may be 100 to 300 nm. If the diameter of the micelle is smaller than the above range, the thickness of the film may be too small to improve the durability. If the diameter of the micelle is larger than the above range, the thickness of the film may become thicker, and thus the purpose of increasing the durability by light feeling of use through a thin film cannot be achieved.

When the film-forming composition is applied to skin or hair, the micelle is broken and the silane and water react with each other to form a film. Specifically, when the film-forming composition is applied to skin or hair, as the micelle breaks in the process of the evaporation and re-emulsification of water, the silane carried in the micelle is brought into contact with and then reacts with water outside the micelle to form a film. Therefore, before use, i.e., application, the silane and water are present separately from each other, so that the composition is easy to store and convenient to use.

In addition, after the film-forming composition is applied to the skin or hair, a further heat treatment may be performed for effective film-forming. In the case of skin, it is possible to apply warm air of a temperature that does not cause burns, to the skin, or to bring the skin into contact with a steam towel to help film-forming. In the case of hair, it is possible to apply heat to the hair using a heat styler (a straightener) or a heat treatment machine used in salons, and it is preferable to apply heat in the range of 80 to 150° C.

Hereinafter, examples, comparative examples and experimental examples will be described to help understand the effects of the present invention. However, it should be noted that the following description is only an example of the contents and effects of the present invention, and the scope and effect of the present invention are not limited thereto.

### EXAMPLE 1

### Preparation of Film-Forming Composition Comprising Emulsion in Which Micelles Carrying Silane and Silicone are Water-Dispersed

5 g of non-ionic surfactant Trideceth-12, 10 g of dimethiconol with weight-average molecular weight of 5000 as silicone-based oil and 2 g of triethoxycaprylylsilane as silane were uniformly mixed at 50° C. Thereafter, 10 g of deionized water was added and stirring was carried out at a high-speed (3600 rpm) for 10 minutes while observing the phase transition point (the point at which the phase transition from the W/O phase to the O/W phase occurs). Then, the temperature was lowered to room temperature, 73 g of deionized water was added, and the mixture was stirred at high speed for 1 hour to prepare an emulsion type film-forming composition in which micelles carrying silane and silicone are water-dispersed.

### COMPARATIVE EXAMPLE 1

### Preparation of Film-Forming Composition Comprising Emulsion in Which Micelles Carrying Silicone are Water-Dispersed

A film-forming composition was prepared in the same method as in Example 1 except that triethoxycaprylylsilane as the silane was not used.

### EXPERIMENTAL EXAMPLE 1

### Measurement of Water Repellency by Measurement of Contact Angle of Hair

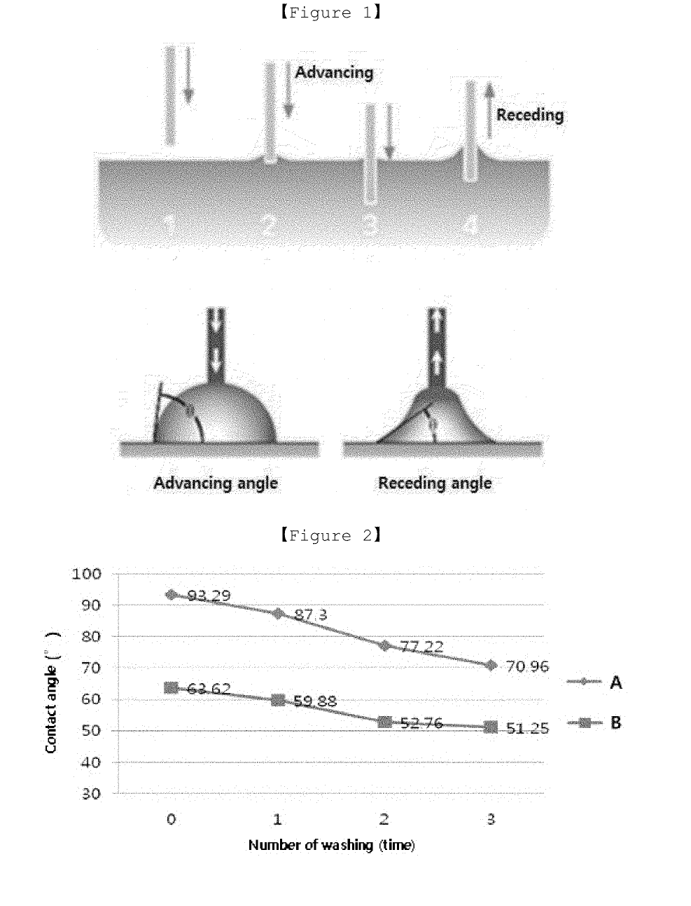

The hair coated with the film-forming composition of Example 1 and the hair coated with the film-forming composition of Comparative Example 1 were washed with shampoo three times in total, and then the contact angle of hair depending on the number of washing times (0, 1, 2 and 3 times) was measured using a measuring device (Process Tensiometer K100), and the measuring process of the contact angle is shown in FIG. 1. Referring to FIG. 1, after the two hairs are installed vertically in the fiber holder, the fiber holder is moved downward at a constant speed so that the hair is immersed in water, and the contact angle of the hair was measured by sensing the weight change of the liquid contacting the hair. The water repellency of the surface of the hair was measured by measuring the contact angle of the hair, and the results are shown in FIG. 2.

As shown in FIG. 2, it can be confirmed that since the contact angle of the hair coated with the film-forming composition of Example 1 was 70.96° even after three time washings, it has excellent water repellency and coating effect on hair surface, as compared with the hair coated with the film-forming composition of Comparative Example 1 showing a contact angle of 51.25°.

### EXPERIMENTAL EXAMPLE 2

### Evaluation of Adsorption Amount and Durability of Film-Forming Composition (Wool Swatch Test)

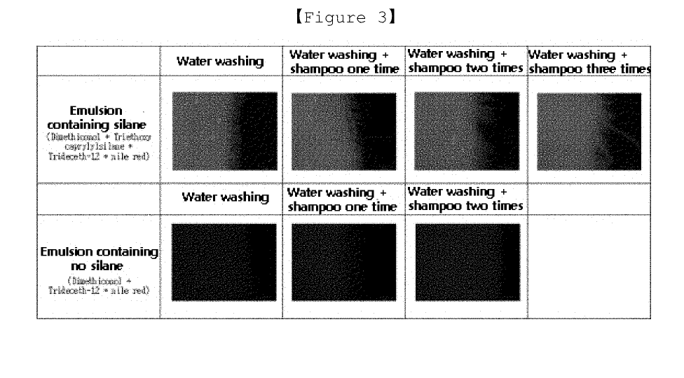

1 g of shampoo was placed on a 5 cm×5 cm wool swatch, 10 g of tap water at 37.5° C. was added, and evenly applied for 1 minute while rubbing lightly with a small force. After rubbing, the wool swatch was thoroughly rinsed with tap water at 37.5° C. at a flow rate of 100 g/s for 30 seconds or more. The above procedure was repeated three times. Thereafter, the wool swatch was immersed in 5% of the film-forming composition of Example 1 which was prepared by using a 0.5% fluorescent dye (nile red (Mw: 318)) solution or 5% of the film-forming composition of Comparative Example 1 which was prepared by using a 0.5% fluorescent dye (nile red (Mw: 318)) solution, for 3 minutes, respectively. After 3 minutes, the wool swatch was removed and then heat-treated at 90° C. for 5 minutes, then rinsed thoroughly with tap water at 37.5 r at a flow rate of 100 g/s for 30 seconds or more, and then air-dried. Then, the degree of coloration was measured with a colorimeter (CHROMA METER CR-100, MINOLTA CAMERA CO., LTD., Japan), and the remaining amount of the composition remaining in the wool swatch was evaluated depending on the number of rinses. The measurement results are shown in FIG. 3.

As shown in FIG. 3, it can be seen that when the composition (Example 1) containing the silane according to the present invention is applied, the change in the amount of adsorption is not large and durability is excellent, even in the case of washing with water and washing with shampoo three times. However, it can be seen that when the composition containing no silane (Comparative Example 1) is applied, most of the components are removed and the adsorption amount of the composition is almost zero. From this, it can be seen that the composition according to the present invention can be easily adsorbed to a substrate by forming a silane film, while the adsorbed components can be retained for a longer time, thereby increasing the durability of the softness of the hair.

### EXPERIMENTAL EXAMPLE 3

### Sensory Evaluation

For Example 1 and Comparative Example 1, twenty women aged 20-30 years were tested for gloss, durability and light feeling of use. The sensory evaluation was performed by the 5-point scaling method and the results were averaged and shown in Table 1 below.

[Evaluation Criteria]

5: very good, 4: good, 3: moderate, 2: poor, 1: very poor

TABLE 1

| Item | Gloss | Durability | Light feeling of use |
|---|---|---|---|
| Example 1 | 4.5 | 4.7 | 4.1 |
| Comparative Example 1 | 3.0 | 2.2 | 4.0 |

As shown in Table 1, it was confirmed that the film-forming composition of Example 1 was superior to the film-forming composition of Comparative Example 1 in terms of gloss and durability.

1. A film-forming composition for skin or hair coating comprising silane and water wherein the silane is water-dispersed.

2. The film-forming composition for skin or hair coating according to claim 1, wherein the film-forming composition has a form in which the micelle carrying silane is water-dispersed and when applied, the silane and the water react with each other to form a film.

3. The film-forming composition for skin or hair coating according to claim 1, wherein the silane is at least one selected from the group consisting of aminopropyltriethoxysilane, hexyltriethoxysilane, perfluorooctyltriethoxysilane, triethoxycaprylylsilane, retinoxytrimethylsilane, stearyltriethoxysilane and stearoxytrimethylsilane.

4. The film-forming composition for skin or hair coating according to claim 1, wherein the silane is contained in an amount of 0.1 to 5 wt. % based on the total weight of the film-forming composition.

5. The film-forming composition for skin or hair coating according to claim 1, wherein the film-forming composition further comprises a silicone-based oil.

6. The film-forming composition for skin or hair coating according to claim 5, wherein the silicone-based oil is contained in an amount of 1 to 40 wt. % based on the total weight of the film-forming composition.

7. The film-forming composition for skin or hair coating according to claim 5, wherein the weight-average molecular weight of the silicone-based oil is 3,000 to 50,000.

8. The film-forming composition for skin or hair coating according to claim 5, wherein the silicone-based oil is at least one selected from the group consisting of dimethiconol, dimethicone, cyclomethicone, cyclophenylmethicone, capryldimethicone, caprylyltrimethicone, caprylylmethicone, cetearylmethicone, hexadecylmethicone, hexylmethicone, laurylmethicone, myristylmethicone, phenylmethicone, stearylmethicone, stearyldimethicone, trifluoropropylmethicone, cetyldimethicone, polyphenylmethylsiloxane, dimethylpolysiloxane, methylphenylpolysiloxane, cyclopentasiloxane, decamethylcyclopentasiloxane, cyclohexasiloxane, cyclohepatsiloxane, cyclotetrasiloxane, cyclotrisiloxane, methyltrimethicone, diphenylsiloxyphenyltrimethicone, caprylylmethicone and phenyltrimethicone.

9. The film-forming composition for skin or hair coating according to claim 1, wherein the film-forming composition further comprises a surfactant, and is an emulsion in which the micelle carrying silane is water-dispersed.

10. The film-forming composition for skin or hair coating according to claim 9, wherein the surfactant is at least one cationic surfactant selected from the group consisting of steartrimoniumchloride, cetrimoniumchloride, behenyltrimethylammonium chloride, cetyltrimethylammonium chloride, stearyltrimethylammonium chloride, distearyldimethylammonium chloride, dicetyldimethylammonium chloride, cocamidopropyldimethylamine, stearamidopropyldimethylamine, behenylamidopropyldimethylamine, oleamidopropyldimethylamine and isostearamidopropyldimethylamine; or at least one non-ionic surfactant selected from the group consisting of Trideceth-10, Trideceth-12, lauryl glucoside, decyl glucoside, PEG-20 glyceryl triisostearate, PEG-15 glyceryl isostearate, polyglyceryl-3 diisostearate and polyglyceryl-10 dioleate.
